(19) United States
(12) Patent Application Publication
(10) Pub. No.: US 2019/0231766 A1
(43) Pub. Date: Aug. 1, 2019

(54) ADMINISTRATION OF ETHYL 3-[(2-{[4-(HEXYLOXYCARBONYL-AMINOIMINOMETHYL)PHENYL-AMINO]METHYL}-1-METHYL-1H-BENZIMIDAZOL-5-CARBONYL)PYRIDIN-2-YLAMINO]PROPIONATE

(71) Applicant: Boehringer Ingelheim International GmbH, Ingelheim am Rhein (DE)

(72) Inventors: Ulrich BRAUNS, Biberach an der Riss (DE); Norbert HAUEL, Schemmerhofen (DE); Peter SIEGER, Mittelbiberach (DE)

(73) Assignee: Boehringer Ingelheim International GmbH, Ingelheim am Rhein (DE)

(21) Appl. No.: 16/380,161

(22) Filed: Apr. 10, 2019

Related U.S. Application Data
(63) Continuation of application No. 15/946,096, filed on Apr. 5, 2018, now abandoned.
Application No. 15/946,096 is a continuation of application No. 14/489,892, filed on Sep. 18, 2014, now abandoned.
Application No. 14/489,892 is a continuation of application No. 10/383,198, filed on Mar. 6, 2003, now abandoned.
(60) Provisional application No. 60/409,762, filed on Sep. 11, 2002.
(60) Provisional application No. 60/421,896, filed on Oct. 29, 2002.

(30) Foreign Application Priority Data
Mar. 7, 2002 (DE) 10209985.5
Sep. 30, 2002 (DE) 10245624.0

(51) Int. Cl. A61K 31/4439; C07D 401/12

(52) U.S. Cl. CPC A61K 31/4439; C07D 401/12

(57) ABSTRACT

The invention relates to a new administration form for the oral application of the active substance ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate and the pharmacologically acceptable salts thereof.

### FIELD OF INVENTION

The invention relates to administration forms for oral applications of prodrugs and in particular prodrugs of the active substance ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate and the pharmacologically acceptable salts thereof.

### BACKGROUND OF THE INVENTION

The invention relates to an administration form for the oral application of the active substance ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate and the pharmacologically acceptable salts thereof. This active substance having the chemical formula

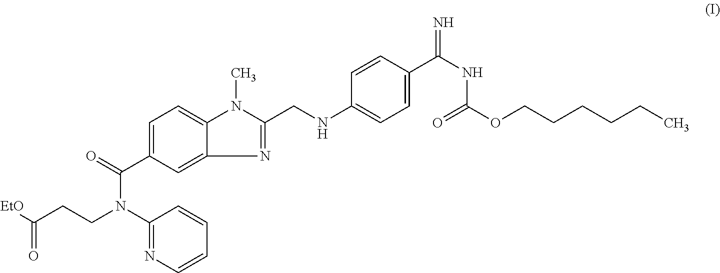 [SMILES: CCCCCCOC(=O)NC(=N)c1ccc(NCc2nc3cc(C(=O)N(CCC(=O)OCC)c4ccccn4)ccc3n2C)cc1]

is already known from WO 98/37075, which discloses compounds with a thrombin-inhibiting effect and the effect of prolonging the thrombin time, under the name 1-methyl-2-[N-[4-(N-n-hexyloxycarbonylamidino)phenyl]-amino-methyl]-benzimidazole-5-yl-carboxylic acid-N-(2-pyridyl)-N-(2-ethoxycarbonylethyl)-amides. The compound of formula I is a double prodrug of the compound

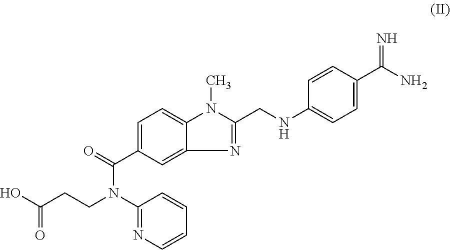 [SMILES: Cn1c(CNc2ccc(C(=N)N)cc2)nc2cc(C(=O)N(CCC(=O)O)c3ccccn3)ccc21]

i.e. the compound of formula I is only converted into the active compound, namely the compound of formula II, after entering the body. The main indication for the compound of chemical formula I is the post-operative prevention of deep-vein thrombosis.

### BRIEF DESCRIPTION OF THE FIGURES

FIG. 1 shows a schematic structure of the pharmaceutical composition.

FIG. 2 shows the bioavailability of BIBR 1048.

### DESCRIPTION OF THE INVENTION

The aim of the invention is to provide an improved formulation for oral use of the compound of formula I (which is also referred to hereinafter as the “active substance”).

Surprisingly it has now been found that the use of pharmaceutically acceptable organic acids with a water solubility of >1 g/250 ml at 20° C., preferably >1 g/160 ml at 25° C., in solid oral preparations leads to a significantly improved formulation of ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate as well as the pharmaceutically acceptable salts thereof.

Pharmaceutically suitable acids for the purposes of this invention are for example tartaric acid, fumaric acid, succinic acid, citric acid, malic acid, glutamic acid and aspartic acid including the hydrates and acid salts thereof. Particularly suitable for the purposes of this invention are tartaric acid, fumaric acid, succinic acid and citric acid.

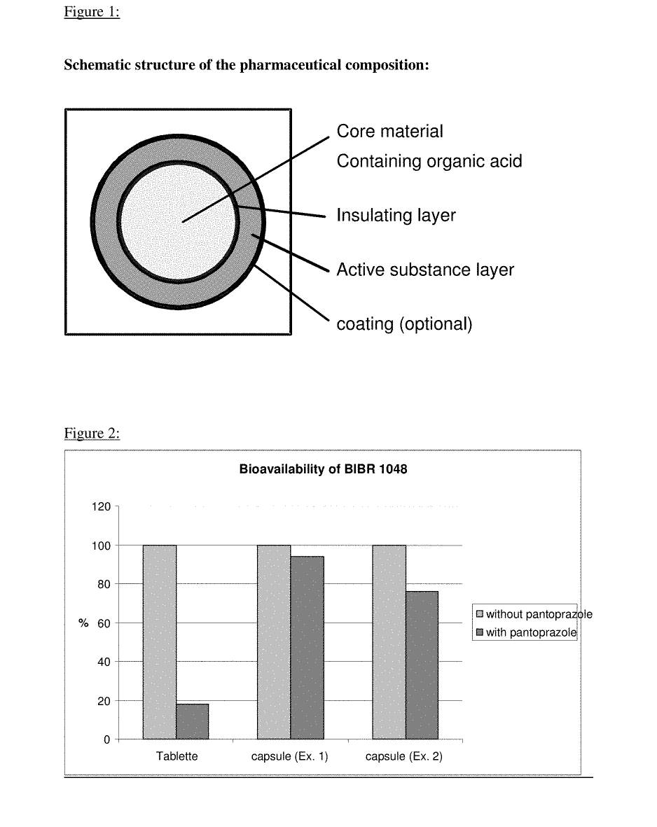

A preferred embodiment of the invention is a multiparticulate preparation in which the individual particles are constructed as in FIG. 1.

FIG. 1 shows the diagrammatic structure of the pharmaceutical composition by means of a section through a pellet suitable for the preparation of the pharmaceutical composition according to the invention. The roughly bead-shaped/spherical core region of this pellet contains/consists of the pharmaceutically acceptable organic acid. Then follows a layer, the so-called insulating layer, which separates the acid core from the layer containing the active substance. The insulating layer is in turn surrounded by the equally spherically shaped layer of active substance which may in turn be enclosed in a coating which increases the abrasion resistance and shelf life of the pellets.

One advantage of the formulation thus constructed is the spatial separation of the organic acid and active substance by the insulating layer. A further advantage of the construction of the pellets as described above is the fact that the organic acid does not go into solution until after the preparation has been taken and then produces an acid microclimate in which the active substance can dissolve.

The core material used is a pharmaceutically acceptable organic acid with a water solubility of >1 g/250 ml at 20° C., such as e.g. tartaric acid, fumaric acid, succinic acid, citric acid, malic acid, glutamic acid and aspartic acid including the hydrates and acid salts thereof, to which a small amount of 1 to 10% by weight, preferably 3 to 6% by weight of a suitable binder is optionally added. The use of a binder may be necessary, for example, if the starting acids are produced by a pan build-up process. If the method used is extrusion or spheronisation, other technological adjuvants such as microcrystalline cellulose will be needed instead of binders. It is also possible to use pure (100%) acid as the starting material if it can be obtained in a sufficiently narrow range of particle sizes. The pharmaceutically acceptable organic acids used are preferably tartaric acid, fumaric acid, succinic acid or citric acid; tartaric acid is particularly preferred. As binder, it is possible to use gum arabic or a partially or totally synthetic polymer selected from among the hydroxypropylcelluloses, hydroxypropylmethylcelluloses, methylcelluloses, hydroxyethylcelluloses, carboxymethylcelluloses, polyvinylpyrrolidone, the copolymers of N-vinylpyrrolidone and vinyl acetate, or combinations of these polymers; gum arabic is preferred. The spherical core material preferably has an average diameter of 0.4-1.5 mm. The content of the pharmaceutically acceptable organic acid is usually between 30 and 100% in the core material, corresponding to an amount of between 20 and 90%, preferably between 20 and 80% in the finished pellet (i.e. in the pharmaceutical composition).

To increase the durability of the finished product it is advantageous to coat the core material before the application of the active substance with an insulating layer based on a water-soluble, pharmaceutically acceptable polymer. Examples of such water-soluble polymers include for example gum arabic or a partially or totally synthetic polymer selected from among the hydroxypropylcelluloses, hydroxypropylmethylcelluloses, methylcelluloses, hydroxyethylcelluloses, carboxymethylcelluloses, polyvinylpyrrolidone, the copolymers of N-vinylpyrrolidone and vinyl acetate, or combinations of these polymers. Gum arabic or a hydroxypropylmethylcellulose is preferably used. If desired, the coating with the water-soluble, pharmaceutically acceptable polymer may be carried out with the addition of suitable plasticisers, separating agents and pigments, such as for example triethylcitrate, tributylcitrate, triacetin, polyethyleneglycols (plasticisers), talc, silicic acid (separating agents), titanium dioxide or iron oxide pigments (pigments).

The active substance layer contains the active substance ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate (BIBR 1048) or one of the pharmaceutically acceptable salts thereof as well as binders and optionally separating agents. A preferred salt of the active substance is the mesylate (methanesulphonate) of the compound of formula I. Suitable binders include for example hydroxypropylcellulose, hydroxypropylmethylcellulose, methylcellulose, hydroxyethylcellulose, carboxymethylcellulose, polyvinylpyrrolidone, copolymers of N-vinylpyrrolidone and vinyl acetate or combinations of these polymers. Preferably, hydroxypropylcellulose or copolymers of N-vinylpyrrolidone and vinyl acetate are used. The addition of separating agents such as e.g. talc or silicic acid serves to prevent the particles from aggregating during the process. The active substance content is 5 to 60%, preferably 10 to 50% of the pharmaceutical composition.

The optional outermost layer, which serves to reduce any increased abrasion during packing into capsules and/or to increase the shelf life, consists of pharmaceutically conventional film-forming agents, plasticisers and optionally pigments. Suitable film-forming agents include for example hydroxypropylcellulose, hydroxypropylmethylcellulose, methylcellulose, polymers and copolymers of acrylic and methacrylic acid and the esters thereof, or combinations of these polymers. Suitable plasticisers include inter alia triethylcitrate, tributylcitrate, triacetin or polyethyleneglycols. The pigments used may be e.g. titanium dioxide or iron oxide pigments. Preferably, the outer coating consists of hydroxypropylmethylcellulose and/or methylcellulose, optionally with the addition of polyethyleneglycols as plasticisers.

The pellets may be prepared by the method described hereinafter:

The acid-containing core material consists either of crystals of the particular organic acid used or, more advantageously, of roughly spherical particles of the desired size containing a large amount of organic acid, which can be produced by methods known and established in pharmaceutical technology. The core material may be produced, in particular, by pan methods, on pelleting plates or by extrusion/spheronisation. Then the core material thus obtained may be divided into fractions of the desired diameter by screening. Suitable core material has an average diameter of 0.4 to 1.5 mm, preferably 0.6 to 0.8 mm.

First, the insulating layer is applied to this acid-containing core material. This can be done by conventional methods, e.g. by applying an aqueous dispersion of the water-soluble, pharmaceutically acceptable polymer, optionally with the addition of plasticisers, separating agents and/or pigments, in a fluidised bed, in coating pans or in conventional film coating apparatus. If necessary the product can then be screened again.

Then the active substance is applied from a dispersion containing binder and optionally separating agent. The volatile dispersant is removed during or after the process by drying. Suitable binders in the dispersion may be for example hydroxypropylcellulose, hydroxypropylmethylcellulose, methylcellulose, hydroxyethylcellulose, carboxymethylcellulose, polyvinylpyrrolidone, copolymers of N-vinylpyrrolidone and vinyl acetate or combinations of these polymers. Preferably, hydroxypropylcellulose or copolymers of N-vinylpyrrolidone and vinyl acetate are used. Suitable separating agents include e.g. talc or silicic acid; preferably, talc is used. The dispersants may be for example ethanol, 2-propanol, acetone or mixtures of these solvents with one another or with water, preferably 2-propanol. The application of active substance to the core material may be carried out by established methods known in pharmaceutical technology, e.g. in coating pans, conventional film coating apparatus or by the fluidised bed method. Then a further screening process may be carried out.

To reduce any increased abrasion during transfer into capsules or to increase the shelf life the system may finally be coated with a coating of a pharmaceutically conventional film forming agent, plasticiser and optionally pigment. This may be done by conventional methods as mentioned earlier in the description of the application of the insulating layer.

When core material with an average diameter of 0.4-1.5 mm is used, the process described above produces pellets containing active substance, which can then be packed into hard capsules, for example. To do this, a number of these units corresponding to the required dosage are packed into hard capsules in a standard capsule filling machine. Suitable hard capsules include, for example, hard gelatine capsules or hard capsules of hydroxypropylmethylcellulose (HPMC); HPMC capsules are preferred. The active substance content of the pharmaceutical composition is 5 to 60%, preferably 10 to 50%; the content of the pharmaceutically acceptable organic acid is usually between 20 and 90%, preferably between 20 and 80%.

Unless otherwise stated, percentages specified are always percent by weight. All the data on the active substance content relate to the active substance base of formula I (not to a specific salt) unless otherwise stated.

Clinical Trials

In preliminary tests on test subjects with conventional tablets containing the compound of formula I it had been established that highly variable plasma levels occurred, with individual cases of malabsorption. The variability of the plasma level patterns is significantly lower after the administration of the compound of formula I as an orally administered solution; there were no cases of malabsorption under these circumstances.

Tests have shown that the compound of formula I dissolves relatively well in water at low pH levels, whereas at pH levels above 5 in accordance with the definition of the European Pharmacopoeia it is virtually insoluble. Therefore the volunteers in one branch of the clinical trials were given pantoprazole, which serves to produce an elevated gastric pH.

For example, the pharmaceutical compositions according to Examples 1 and 2 were tested for their bioavailability by comparison with a conventional tablet.

To do this, the formulation prepared according to Example 1 containing 50 mg of active substance base per capsule was clinically tested for its bioavailability on a total of 15 volunteers. In one branch of the treatment, the volunteers were given the composition by mouth (=orally) on an empty stomach without any pre-treatment. In another branch of the treatment the same volunteers were pre-treated, prior to the oral administration of the composition, with 40 mg of pantoprazole b.i.d. (=twice a day) for three days by mouth to increase the gastric pH; the treatment with pantoprazole was continued during the administration of the formulation according to the invention.

The degree of absorption was determined by measuring the quantity of active metabolite of formula II excreted in the urine.

The relative bioavailability after pre-treatment with pantoprazole was 94% on average compared with administration without any pre-treatment.

Under comparable conditions of administration, the relative bioavailability (based on the area under the plasma concentration/time curve) of a tablet containing 50 mg of active substance, developed and produced according to the prior art and containing no water-soluble organic acid, after corresponding pre-treatment with pantoprazole, is 18%. Table I shows the precise composition of the tablet used:

TABLE I

| | Ingredient | mg/tablet |
|---|---|---|
| Core | mesylate of the compound of form. I | 57.7 |
| | lactose monohydrate | 58.0 |
| | microcrystalline cellulose | 48.3 |
| | crospovidone | 3.4 |
| | magnesium stearate | 2.6 |
| Film coating | polyethyleneglycol 6000 | 0.56 |
| | titanium dioxide | 0.80 |
| | talc | 0.64 |
| | hydroxypropylmethylcellulose | 1.92 |
| | iron oxide yellow | 0.08 |
| | Total | 174.0 |

The relative bioavailability was thus improved by about a factor of 5 by using the formulation according to the invention.

The formulation prepared according to Example 2 containing 50 mg of active substance base per capsule was also clinically tested for its bioavailability on a total of 15 volunteers.

In one branch of the treatment, the volunteers were given the composition by mouth on an empty stomach without any pre-treatment. In another branch of the treatment the same volunteers were pre-treated, prior to the oral administration of the composition, with 40 mg of pantoprazole b.i.d. for three days by mouth to increase the gastric pH; the treatment with pantoprazole was continued during the administration of the formulation according to the invention.

The degree of absorption was determined by measuring the quantity of the active metabolite of formula II excreted in the urine.

The relative bioavailability after pre-treatment with pantoprazole was 76% on average compared with administration without any pre-treatment.

Under comparable conditions of administration, the relative bioavailability (based on the area under the plasma concentration/time curve) of a tablet containing 50 mg of active substance, developed and produced according to the prior art and containing no water-soluble organic acid, after corresponding pre-treatment with pantoprazole, is 18%. Table II shows the precise composition of the tablet used:

TABLE II

| | Ingredient | mg/tablet |
|---|---|---|
| Core | mesylate of the compound of form. I | 57.7 |
| | lactose monohydrate | 58.0 |
| | microcrystalline cellulose | 48.3 |
| | crospovidone | 3.4 |
| | magnesium stearate | 2.6 |
| Film coating | polyethyleneglycol 6000 | 0.56 |
| | titanium dioxide | 0.80 |
| | talc | 0.64 |
| | hydroxypropylmethylcellulose | 1.92 |
| | iron oxide yellow | 0.08 |
| | Total | 174.0 |

The relative bioavailability of the active substance compared with conventional formulations was thus improved by about a factor of 4 by using the formulation according to the invention. The bioavailability of the two formulations according to the invention compared with the tablet described above with and without the simultaneous administration of pantoprazole is graphically illustrated in FIG. 2.

The clinical trials show another advantage of the preparation according to the invention containing the compound of formula I, which is that it ensures adequate bioavailability of the active substance, better than that of a conventional pharmaceutical preparation and largely independent of the gastric pH, it reduces fluctuations in the bioavailability of the active substance and it prevents malabsorption. Another advantageous property of the pharmaceutical composition according to the invention is the fact that it is suitable for all patients, i.e. including those in whom the gastric pH is increased by normal physiological variability, by disease or by co-medication with drugs which raise the gastric pH.

The dosage for oral use is expediently 25 to 300 mg of the active substance base (per capsule), preferably 50 to 200 mg, most preferably 75 to 150 mg of the active substance base, in each case once or twice a day.

The preferred ratio of acid to active substance is about 0.9:1 to about 4:1, most preferably between about 1:1 and 3:1. Preferably, at least one equivalent of acid is used per mol of the compound of formula I. The upper limit of about 4:1 (acid to active substance) is generally determined by the maximum acceptable size of the preparation in the desired dosages (number of pellets per capsule).

The Examples that follow are intended to illustrate the invention:

### Example 1

| | percentage composition | | | | | |
|---|---|---|---|---|---|---|
| | | | active | | per | per |
| | core | insulating | substance | | capsule | capsule |
| | material | layer | layer | total | [mg] | [mg] |
| tartaric acid | 61.3 | — | — | 61.3 | 176.7 | 353.4 |
| gum arabic | 3.1 | 2.8 | | 5.9 | 17.0 | 34.0 |
| talc | — | 5.6 | 3.2 | 8.8 | 25.4 | 50.7 |
| hydroxypropylcellulose | — | — | 4.0 | 4.0 | 11.5 | 23.1 |
| active substance (mesylate | — | — | 20.0 | 20.0 | 57.7* | 115.3** |
| of the compound of | | | | | | |
| formula I) | | | | | | |
| total | | | | 100.0 | 288.3 | 576.5 |

*corresponds to 50 mg of the compound of formula 1 (active substance base)
**corresponds to 100 mg of the compound of formula 1 (active substance base)

a) Production of Core Material Containing Tartaric Acid

Composition:

| | | |
|---|---|---|
| gum arabic | 1 | part by weight |
| tartaric acid | 20 | parts by weight |

1 part by weight of gum arabic is dissolved In 4 parts by weight of purified water at 50° C. with stirring. Then 5 parts by weight of tartaric acid are dissolved in this solution with stirring.

8.3 parts by weight of tartaric acid crystals with an average particle size of 0.4 to 0.6 mm are placed in a suitable coating apparatus fitted with an air inlet and exhaust, and the pan is set in rotation. At an air inlet temperature of 60°-80° C. the tartaric acid crystals are sprayed at intervals with the solution of tartaric acid and gum arabic and sprinkled with a total of 6.7 parts by weight of powdered tartaric acid, so that roughly spherical particles are formed.

The spherical tartaric acid core material is then dried in the rotating pan at an air inlet temperature of 60°-80° C.

The core material is fractionated using a tumbler screening machine with perforated plates with a nominal mesh size of 0.6 and 0.8 mm. The product fraction between 0.6 and 0.8 mm is used in the rest of the process.

b) Insulation of the Core Material Containing Tartaric Acid

Composition:

| | | |
|---|---|---|
| core material containing tartaric acid | 23 | parts by weight |
| gum arabic | 1 | part by weight |
| talc | 2 | parts by weight |

1 part by weight of gum arabic is dissolved in a mixture of 6.7 parts by weight of 96% ethanol and 13.5 parts by weight of purified water with stirring. Then 2 parts by weight of talc are dispersed in the solution with stirring.

In a fluidised bed processing apparatus, 23 parts by weight of core material containing tartaric acid are sprayed at an air inlet temperature of 35°-40° C. with the dispersion of gum arabic and talc by the under-bed spraying process.

The insulated core material containing tartaric acid is then dried in the circulating air drier at 40° C. for 8 hours.

To remove any lumps the dried insulated core material containing tartaric acid is screened through a screen with a nominal mesh size of 1.0 mm. The fraction of material with a particle size of <1 mm is further processed.

c) Production of the Active Substance Layer

Composition:

| | | |
|---|---|---|
| insulated core material containing tartaric acid | 91 | parts by weight |
| hydroxypropylcellulose | 5 | parts by weight |
| talc | 4 | parts by weight |
| active substance (mesylate of BIBR 1048) | 25 | parts by weight |

Hydroxypropylcellulose is dissolved in 168 parts by weight of 2-propanol with stirring and then the active substance and talc are dispersed in this solution with stirring.

In a fluidised bed processing apparatus, 91 parts by weight of insulated core material containing tartaric acid are sprayed at an air inlet temperature of 20°-30° C. with the dispersion containing the active substance by the under-bed spraying process.

The pellets containing the active substance are then dried in the circulating air drier at 35° C. for 8 hours.

To remove any lumps the pellets containing the active substance are screened through a screen with a nominal mesh size of 1.25 mm. The fraction of material with a particle size of <1.25 mm is further processed.

d) Packing into Capsules

A quantity of active substance pellets containing in each case 50 or 100 mg of active substance base is packed into size 1 or size 0 elongated hard gelatine capsules or HPMC capsules by means of a capsule filling machine.

### Example 2

| | percentage composition | | | | | |
|---|---|---|---|---|---|---|
| | | | active | | per | per |
| | core | insulating | substance | | capsule | capsule |
| | material | layer | layer | total | [mg] | [mg] |
| tartaric acid | 38.5 | — | — | 38.5 | 55.5 | 166.5 |
| gum arabic | 1.9 | 1.7 | | 3.6 | 5.2 | 15.6 |
| talc | — | 3.5 | 6.4 | 9.9 | 14.3 | 42.8 |
| hydroxypropylcellulose | — | — | 8.0 | 8.0 | 11.5 | 34.6 |
| active substance (mesylate | — | — | 40.0 | 40.0 | 57.7* | 173.0** |
| of the compound of | | | | | | |
| formula I) | | | | | | |
| total | | | | 100.0 | 144.2 | 432.5 |

*corresponds to 50 mg of the compound of formula 1 (active substance base)
**corresponds to 150 mg of the compound of formula 1 (active substance base)

a) Production of Core Material Containing Tartaric Acid

Composition:

| | | |
|---|---|---|
| gum arabic | 1 | part by weight |
| tartaric acid | 20 | parts by weight |

1 part by weight of gum arabic is dissolved in 4 parts by weight of purified water at 50° C. with stirring. Then 5 parts by weight of tartaric acid are dissolved in this solution with stirring.

8.3 parts by weight of tartaric acid crystals with an average particle size of 0.4 to 0.6 mm are placed in a suitable coating apparatus fitted with an air inlet and exhaust, and the pan is set in rotation. At an air inlet temperature of 60°-80° C. the tartaric acid crystals are sprayed at intervals with the solution of tartaric acid and gum arabic and sprinkled with a total of 6.7 parts by weight of powdered tartaric acid, so that roughly spherical particles are formed.

The spherical tartaric acid core material is then dried in the rotating pan at an air inlet temperature of 60°-80° C.

The core material is fractionated using a tumbler screening machine with perforated plates with a nominal mesh size of 0.6 and 0.8 mm. The product fraction between 0.6 and 0.8 mm is used in the rest of the process.

b) Insulation of the Core Material Containing Tartaric Acid

Composition:

| | | |
|---|---|---|
| core material containing tartaric acid | 23 | parts by weight |
| gum arabic | 1 | part by weight |
| talc | 2 | parts by weight |

1 part by weight of gum arabic is dissolved in a mixture of 6.7 parts by weight of 96% ethanol and 13.5 parts by weight of purified water with stirring. Then 2 parts by weight of talc are dispersed in the solution with stirring.

In a fluidised bed processing apparatus, 23 parts by weight of core material containing tartaric acid are sprayed at an air inlet temperature of 35°-40° C. with the dispersion of gum arabic and talc by the under-bed spraying process.

The insulated core material containing tartaric acid is then dried in the circulating air drier at 40° C. for 8 hours.

To remove any lumps the dried insulated core material containing tartaric acid is screened through a screen with a nominal mesh size of 1.0 mm. The fraction of material with a particle size of <1 mm is further processed.

c) Production of the Active Substance Layer

Composition:

| | | |
|---|---|---|
| insulated core material containing tartaric acid | 57 | parts by weight |
| hydroxypropylcellulose | 10 | parts by weight |
| talc | 8 | parts by weight |
| active substance (mesylate of BIBR 1048) | 50 | parts by weight |

Hydroxypropylcellulose is dissolved in 335 parts by weight of 2-propanol with stirring and then the active substance and talc are dispersed in this solution with stirring.

In a fluidised bed processing apparatus, 91 parts by weight of insulated core material containing tartaric acid are sprayed at an air inlet temperature of 20°-30° C. with the dispersion containing the active substance by the under-bed spraying process.

The pellets containing the active substance are then dried in the circulating air drier at 35° C. for 8 hours.

To remove any lumps the pellets containing the active substance are screened through a screen with a nominal mesh size of 1.25 mm. The fraction of material with a particle size of <1.25 mm is further processed.

d) Packing into Capsules

A quantity of active substance pellets containing in each case 50 or 150 mg of active substance base is packed into size 2 or size 0 hard gelatine capsules or HPMC capsules by means of a capsule filling machine.

### Example 3

### Preparation of ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate methanesulphonate

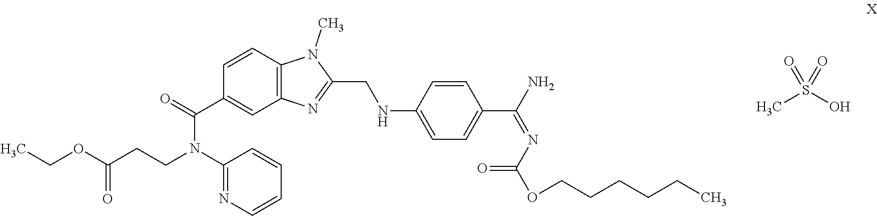

A solution of 5.0 mmol of methanesulphonic acid in 25 ml ethyl acetate was added dropwise, with stirring, to a solution of 3139 mg (5.0 mmol) of ethyl 3-[(2-{[4-(hexyloxycarbonylamino-imino-methyl)-phenylamino]-methyl}-1-methyl-1H-benzimidazole-5-carbonyl)-pyridin-2-yl-amino]-propionate base (prepared as described in WO 98/37075) in 250 ml ethyl acetate, at ambient temperature. After a few minutes the product began to crystallise out. It was stirred for another hour at ambient temperature and then for one more hour while cooling with ice, the precipitate was suction filtered, washed with about 50 ml of ethyl acetate and 50 ml of diethyl ether and dried at 50° C. in a circulating air drier.

Yield: 94% of theory

melting point: 178-179° C.

C₃₄H₄₁N₇O₅×CH₄SO₃ (723.86)

Elemental analysis:

| calc.: | C 58.07% | H 6.27% | N 13.55% | S 4.43% |
|---|---|---|---|---|
| found: | 58.11% | 6.30% | 13.50% | 4.48% |

We claim:

1. A method to inhibit thrombin in a patient in need thereof comprising administering a methanesulphonate salt of ethyl 3-[(2-{[4-(hexyloxycarbonyl-amino-imino-methyl)-phenylamino]-methyl}-1-methyl-1H-benzimidazol-5-carbonyl)-pyridin-2-yl-amino]-propionate to the patient such that the patient receives an oral dose from 50 mg to 200 mg of ethyl 3-[(2-{[4-(hexyloxycarbonyl-amino-imino-methyl)-phenylamino]-methyl}-1-methyl-1H-benzimidazol-5-carbonyl)-pyridin-2-yl-amino]-propionate once or twice a day.

2. A method to inhibit thrombin in a patient in need thereof comprising administering a methanesulphonate salt of ethyl 3-[(2-{[4-(hexyloxycarbonyl-amino-imino-methyl)-phenylamino]-methyl}-1-methyl-1H-benzimidazol-5-carbonyl)-pyridin-2-yl-amino]-propionate to the patient such that the patient receives an oral dose from 75 mg to 150 mg of ethyl 3-[(2-{[4-(hexyloxycarbonyl-amino-imino-methyl)-phenylamino]-methyl}-1-methyl-1H-benzimidazol-5-carbonyl)-pyridin-2-yl-amino]-propionate once or twice a day.

3. A method to inhibit thrombin in a patient in need thereof comprising administering a methanesulphonate salt of ethyl 3-[(2-{[4-(hexyloxycarbonyl-amino-imino-methyl)-phenylamino]-methyl}-1-methyl-1H-benzimidazol-5-carbonyl)-pyridin-2-yl-amino]-propionate to the patient such that the patient receives an oral dose of 150 mg of ethyl 3-[(2-{[4-(hexyloxycarbonyl-amino-imino-methyl)-phenylamino]-methyl}-1-methyl-1H-benzimidazol-5-carbonyl)-pyridin-2-yl-amino]-propionate once or twice a day.

4. The methanesulphonate salt of ethyl 3-[(2-{[4-(hexyloxycarbonyl-amino-imino-methyl)-phenylamino]-methyl}-1-methyl-1H-benzimidazol-5-carbonyl)-pyridin-2-yl-amino]-propionate.

5. A pharmaceutical composition comprising the methanesulphonate salt of ethyl 3-[(2-{[4-(hexyloxycarbonyl-amino-imino-methyl)-phenylamino]-methyl}-1-methyl-1H-benzimidazole-5-carbonyl)-pyridin-2-yl-amino]-propionate.
